(19) United States
(12) Patent Application Publication
(10) Pub. No.: US 2025/0387602 A1
(43) Pub. Date: Dec. 25, 2025

(54) ORAL DRUG DELIVERY DEVICE WITH EXPANDING ARMS

(71) Applicant: Eli Lilly and Company, Indianapolis, IN (US)

(72) Inventors: Gary David KIPLING, Royston (GB); Emma Louise LINDSEY, Cambridge (GB)

(21) Appl. No.: 19/108,001

(22) PCT Filed: Sep. 18, 2023
(86) PCT No.: PCT/US2023/074436

§ 371 Date: Feb. 28, 2025

Related U.S. Application Data
(60) Provisional application No. 63/407,809, filed on Sep. 19, 2022.

(51) Int. Cl. A61M 31/00; A61M 5/142; A61M 5/145; A61M 5/158; A61M 5/168

(52) U.S. Cl. CPC A61M 31/00; A61M 5/14276; A61M 5/1454; A61M 5/158; A61M 5/16813; A61M 2005/14284; A61M 2005/14513; A61M 2210/106

(57) ABSTRACT

The present disclosure provides a drug delivery device. The drug delivery device is taken orally by a patient, and then activates within the gastrointestinal (GI) tract of the patient. Upon activation, arms of the drug delivery device expand, and penetrating tips penetrate the GI tract walls. A driver then drives a plunger within the drug delivery device, pushing a drug through the penetrating tips and through the GI tract walls of the patient. After a period of time, part of the drug delivery device dissolves and the drug delivery device passes through the GI tract.

### FIELD OF THE DISCLOSURE

The present disclosure relates to an oral drug delivery device. More specifically, the present disclosure relates to an oral drug delivery device with expanding arms that activates within the small intestine to deliver a drug through the gastrointestinal wall.

### BACKGROUND OF THE DISCLOSURE

For patients being treated with a drug or some other biologically active compound, it is often most convenient to receive the drug orally. However, the nature of some compounds prevents them from maintaining their activity once consumed. For example, some compounds are denatured, digested, or otherwise deactivated once placed in the environment of the gastrointestinal (GI) system. Additionally, some compounds have low rates of diffusion into the bloodstream from the GI system, which can prevent adequate dosages from being delivered to the patient. For compounds with these characteristics, patients often receive the compounds through injection, which is painful and inconvenient. Accordingly, it is desirable to develop an oral drug delivery device that can successfully deliver a drug that would otherwise be ineffective when taken orally.

### SUMMARY

The present disclosure provides a drug delivery device. The drug delivery device is taken orally by a patient, and then activates within the gastrointestinal (GI) tract of the patient. Upon activation, arms of the drug delivery device expand, and penetrating tips penetrate the GI tract. A driver then drives a plunger within the drug delivery device, pushing a drug through the penetrating tips and into the GI tract walls of the patient. After a period of time, at least a portion of the drug delivery device dissolves or biodegrades and the drug delivery device passes through and exits the GI tract.

In an exemplary embodiment, a drug delivery device includes a capsule configured to degrade within a gastrointestinal tract of a patient. A drug delivery mechanism is disposed within the capsule and is configured to interact with a gastrointestinal tract wall of the patient. The drug delivery mechanism includes a plurality of deployable arms, a plurality of interfacing ends coupled to the plurality of deployable arms, and a plurality of drug delivery channels. The plurality of interfacing ends are in fluid communication with the plurality of drug delivery channels. A drug housing is in fluid communication with the drug delivery mechanism and is configured to contain a volume of a drug. A deployment mechanism is coupled to the drug housing and is configured to move the plurality of deployable arms from an initial configuration to a deployed configuration following a degradation of the capsule. A driving mechanism is coupled to the drug housing and includes a driver and a stopper. The driver is configured to move the stopper to force the drug through the plurality of drug delivery channels and the plurality of interfacing ends.

In another embodiment, a drug delivery device includes a capsule configured to degrade within a gastrointestinal tract of a patient. A drug delivery mechanism is disposed within the capsule and is configured to interact with a gastrointestinal tract wall of the patient. The drug delivery mechanism includes a base and a plurality of deployable arms coupled to the base. Each deployable arm includes a drug delivery channel and an interfacing end in fluid communication with the drug delivery channel. The interfacing end of each deployable arm is configured to interact with a gastrointestinal tract wall of the patient. A drug housing is in fluid communication with the drug delivery mechanism and is configured to contain a volume of a drug. A deployment mechanism includes a collar configured to engage the drug delivery mechanism. A driving mechanism is coupled to the drug housing and to the deployment mechanism. The driving mechanism is operative to move the collar of the deployment mechanism into engagement with the plurality of deployable arms to deploy the plurality of deployable arms. The driving mechanism is further operative to force delivery of the drug through the plurality of drug delivery channels.

### BRIEF DESCRIPTION OF THE DRAWINGS

The above-mentioned and other features and advantages of this disclosure, and the manner of attaining them, will become more apparent and the invention itself will be better understood by reference to the following description of embodiments of the invention taken in conjunction with the accompanying drawings, wherein:

FIG. 1 is a perspective view of an exemplary embodiment of a drug delivery device according to the present disclosure in an initial configuration;

FIG. 2 is a perspective view of the drug delivery device of FIG. 1 in a deployed configuration;

FIG. 3 is an exploded perspective view of the drug delivery device of FIG. 1;

FIG. 4 is a side view of the drug delivery device of FIG. 1 in the deployed configuration;

FIG. 5 is a side sectional view of the drug delivery device of FIG. 1 in the deployed configuration;

FIG. 6 is a front view of the drug delivery device of FIG. 1 in the deployed configuration;

FIG. 7 is a detail perspective view of a delivery member of the drug delivery device of FIG. 1;

FIG. 8 is a detail side view of the delivery member of FIG. 7;

FIG. 9 is a detail side view of a one-way motion mechanism of the drug delivery device of FIG. 1;

FIG. 10 is a side sectional view of the drug delivery device of FIG. 1 in an intermediate configuration;

FIG. 11 is another side view of the drug delivery device of FIG. 1 in the deployed configuration; and

FIG. 12 is another side sectional view of the drug delivery device of FIG. 1 in the deployed configuration.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

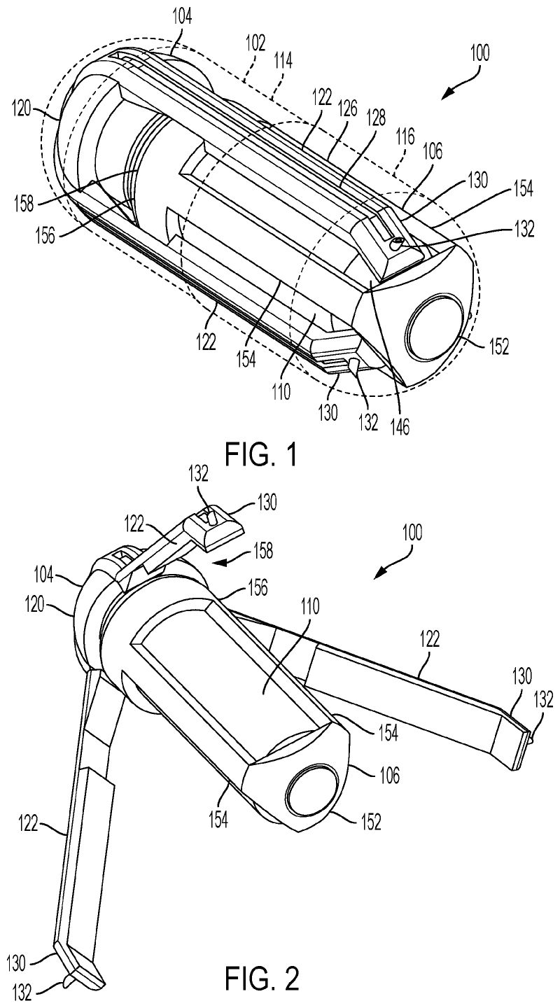

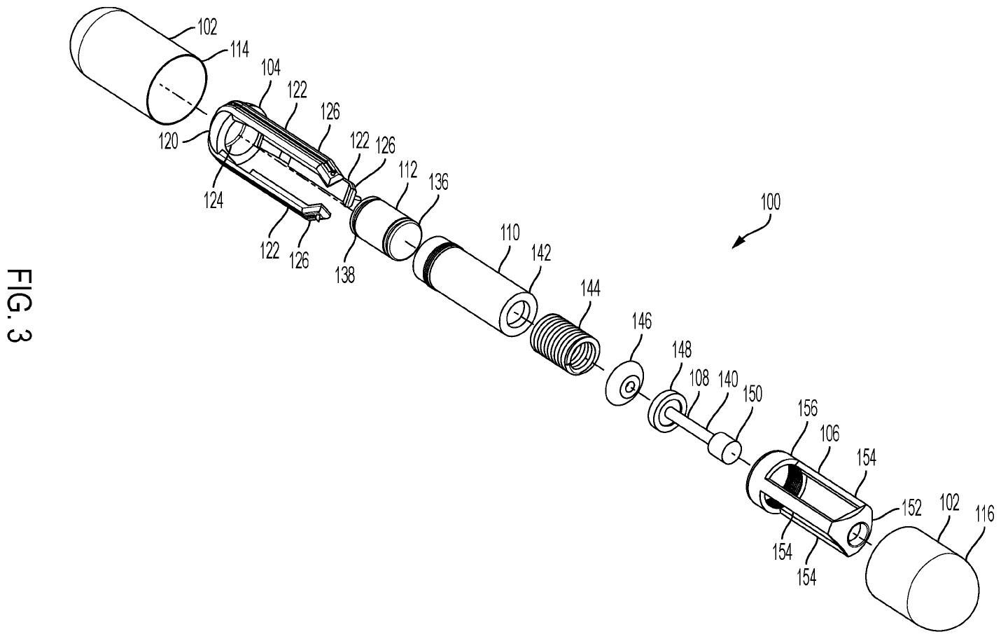

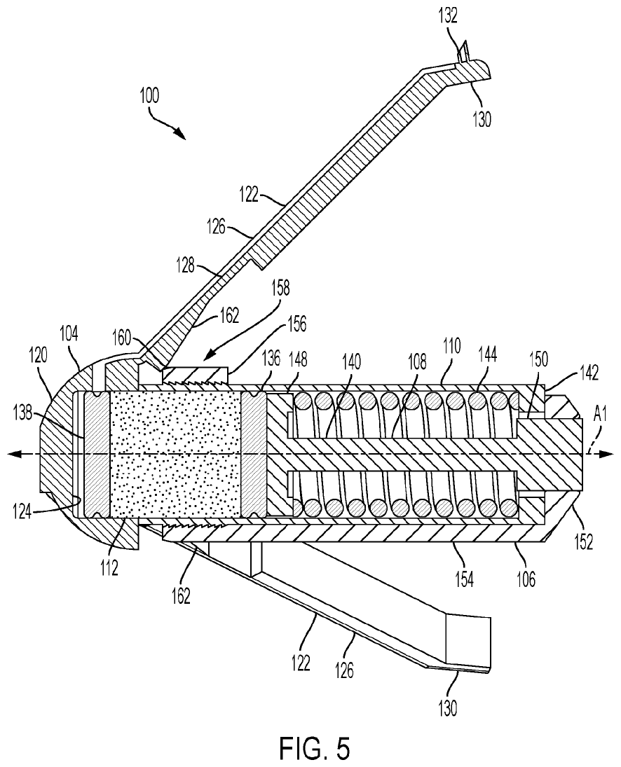

First referring to FIGS. 1-6, an exemplary drug delivery device 100 is illustrated. FIG. 1 illustrates drug delivery device 100 in an initial configuration, and FIGS. 2-6 illustrate drug delivery device 100 in a deployed configuration. Drug delivery device 100 includes a capsule 102 (FIGS. 1 and 3), a delivery mechanism 104, a deployment mechanism 106, a driving mechanism 108 (FIGS. 3 and 5), and a drug housing 110. Drug housing 110 may also be referred to as a cartridge. When drug delivery device 100 is fully assembled and the initial configuration (FIG. 1), capsule 102 encloses and houses delivery mechanism 104, deployment mechanism 106, driving mechanism 108, and drug housing 110. As is discussed in greater detail herein, drug delivery device 100 is taken orally by a patient. Upon entering a portion (e.g., the small intestine) of the gastrointestinal (GI) tract of the patient, capsule 102 dissolves or otherwise degrades. As illustrated in FIG. 2, deployment mechanism 106 then causes deployment of delivery mechanism 104. More specifically, deployment mechanism 106 causes the delivery mechanism 104 to interact with the interior wall of the GI tract and secure drug delivery device 100 in place in the GI tract. Once delivery mechanism 104 has interacted with the patient's GI tract, driving mechanism 108 causes delivery of a drug 112 (FIGS. 3 and 5) from drug housing 110 to the patient through the GI tract wall. In the illustrated embodiment, once deployed the penetrating tips of delivery mechanism 104 are positioned in the GI tract wall such that drug 112 is delivered into the GI tract wall. Once drug 112 has been delivered, at least part of drug delivery device 100 dissolves, and drug delivery device 100 passes through and exits the GI tract.

In an exemplary embodiment, at least a portion of the capsule 102 degrades as the environment around the drug delivery device 100 changes pH, for example when leaving the acidic stomach and entering the relatively alkaline small intestine. In an illustrative embodiment of FIG. 1, capsule 102 includes two components, specifically a first, rear capsule portion 114 and a second, front capsule portion 116. First capsule portion 114 and second capsule portion 116 may be coupled together to form capsule 102. Capsule 102 may be at least partially constructed of one or more dissolvable or otherwise degradable materials. In some embodiments, first capsule portion 114 and second capsule portion 116 are coupled together by an enteric seam 118 (FIG. 1) that degrades in the GI tract to permit capsule 102 to separate and release the remainder of device 100 into the GI tract.

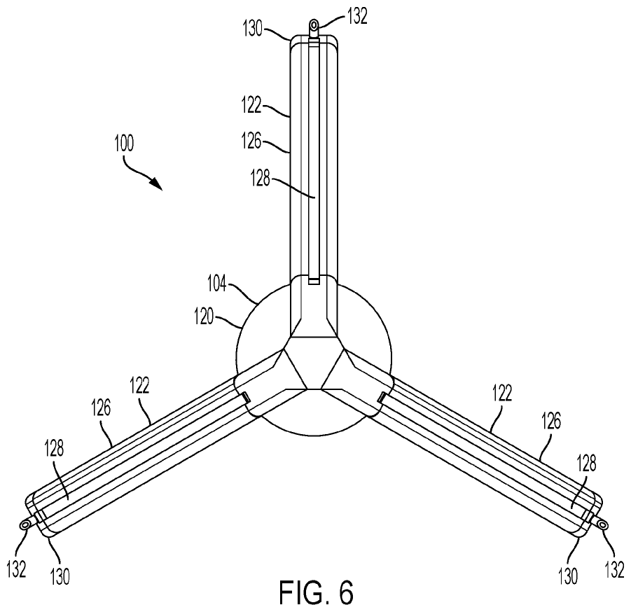

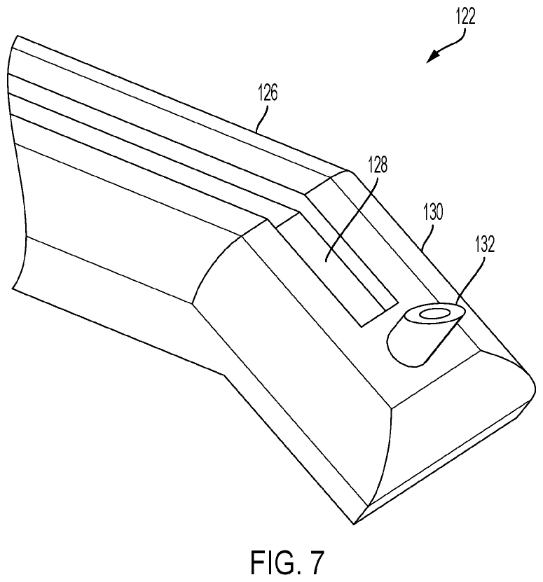

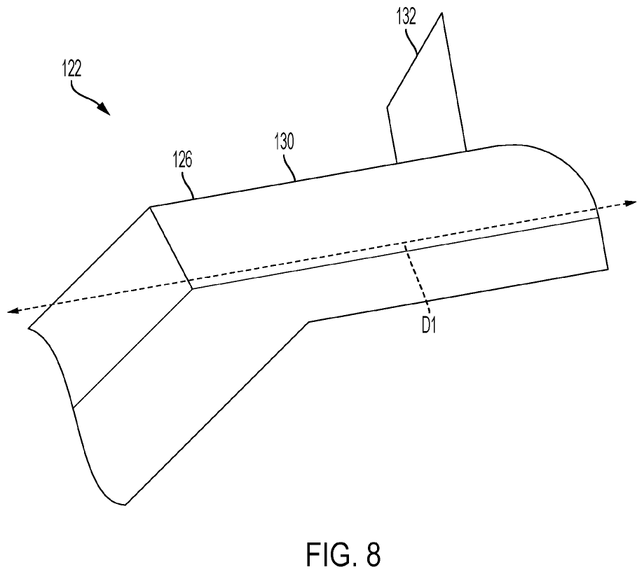

Delivery mechanism 104 fits within capsule 102 when drug delivery device 100 is assembled. In the illustrated embodiment, delivery mechanism 104 comprises a delivery base 120, a plurality of delivery members 122 extending from delivery base 120, one or more membranes (not shown), and a central bore 124 (FIGS. 3 and 5) extending through delivery base 120. With specific reference to FIG. 6 and additional reference to FIGS. 7 and 8, each delivery member 122 comprises a deployable arm 126, a delivery channel 128, an interfacing end 130, and a penetrating tip 132. In the illustrated embodiment, three delivery members 122 are coupled to and extend from delivery base 120, but in other embodiments any number of delivery members 122 may be used. In some embodiments, interfacing ends 130 and/or other components of delivery members 122 carry or otherwise include an adhesive (e.g., mucoadhesive) or fastener (e.g., hook(s)) to facilitate securing device 100 to the interior wall of the GI tract.

Delivery mechanism 104 facilitates drug flow from central bore 124 through delivery channels 128 to interfacing ends 130. Delivery channels 128 extend into and are in fluid communication with central bore 124 and extend along deployable arms 126. In one embodiment, delivery channels 128 are formed as exposed grooves in an outer surface of deployable arms 126. The one or more membranes (not shown) are adhered to the surface of deployable arms 126 in order to enclose and seal delivery channels 128. The membrane(s) may be adhered to the surface of the deployable arms 126 through an adhesive, welding (heat, UV, laser, ultrasonic, solvent, friction, injection, high frequency, etc.), mechanical connections, or any other connective means. Use of the membrane may simplify fabrication (for example, molding, cutting) of channel 128 into the outer surface of deployable arms 126. In other embodiments, delivery channels 128 may be a separate component, such as a tube, coupled to a portion of delivery mechanism 104. Furthermore, delivery channels 128 may be located entirely within deployable arms 126 such that the interior of delivery channels 128 is fully enclosed (for example, a bore through deployable arm 126).

Referring to FIGS. 7 and 8, interfacing ends 130 are located near the end of each deployable arm 126. Each interfacing end 130 includes a penetrating tip 132, which pierces or penetrates the GI tract wall of the patient in the deployment configuration. In an exemplary embodiment, tips 132 are at least partially formed of a material that begins to degrade or dissolve upon exposure to the small intestine fluids. In the illustrated embodiment, after an amount of time has passed following deployment sufficient for completion of delivery, penetrating tips 132 degrade sufficiently to break free from the GI tract, thereby allowing drug delivery device 100 to continue along the GI tract and eventually exit the patient.

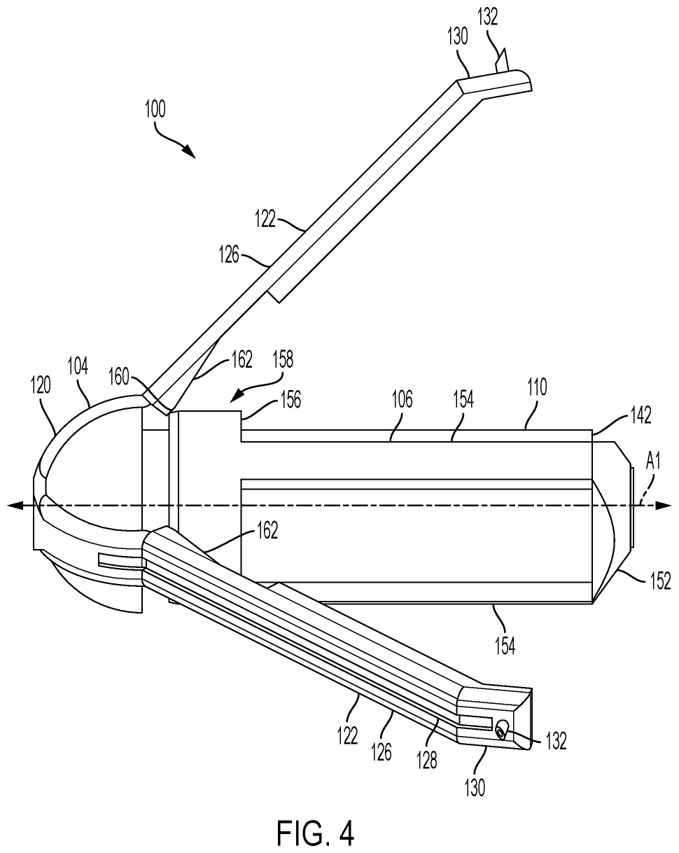

In the illustrated embodiment, penetrating tip 132 has a beveled shape similar to that of a hypodermic needle. In other embodiments, penetrating tip 132 may comprise a piercing tip with a fluid outlet located below the piercing tip (for example, in a side surface of the penetrating tip 132) to reduce the likelihood of blockage in the delivery of drug 112. Furthermore, each delivery member 122 may comprise any number of penetrating tips 132, including arrays of microneedles. In some embodiments and as illustrated, when the deployable arms 126 are deployed the interfacing ends 130 are extended along an oblique direction D1 (FIG. 8) that is obliquely disposed relative to a longitudinal axis A1 of drug delivery device 100 (FIG. 4). In some embodiments, penetrating tips 132 are approximately perpendicular to oblique direction D1.

In some embodiments, penetrating tips 132 may form part of the penetration assemblies and corresponding sockets or receiving bores described in International Application Publication No. WO2022/060817, the disclosure of which is hereby incorporated by reference for all purposes. In an alternative embodiment, interfacing ends 130 may include a liquid jet delivery mechanism for delivering the fluid through the GI tract, as described in the previously referenced International Application Publication No. WO2022/060817.

Referring again to FIGS. 1-6, in the illustrated embodiment, delivery base 120 and delivery members 122 of delivery mechanism 104 are constructed as a single piece. In an exemplary embodiment, delivery mechanism 104 is constructed of a polymer, but in other embodiments delivery mechanism 104 can be constructed of a metal, or any other biocompatible material. Exemplary polymers include polyglycolic acid, polylactic acid, polycaprolactone, and copolymers and blends thereof that may include polyethylene glycol. In other embodiments, delivery mechanism 104 may be constructed of multiple parts connected through one or more coupling members (not shown). Examples of such constructions and coupling members are provided in the previously referenced International Application Publication No. WO2022/060817.

With specific reference to FIG. 5, drug housing 110 is in fluid communication with delivery mechanism 104. Drug housing 110 holds a volume of drug 112, generally in a liquid or otherwise flowable form. In an exemplary embodiment, drug 112 is a compound that typically has less efficacy when taken through standard oral delivery and digestion, such as peptides or proteins like insulin, as well as vaccines or gene-based drugs. In other embodiments, drug 112 may be any biologically active compound to be administered to the patient. In exemplary embodiments, drug 112 includes one or more therapeutic agents including but not limited to insulins, insulin analogs such as insulin lispro or insulin glargine, insulin derivatives, GLP-1 receptor agonists such as dulaglutide or liraglutide, glucagon, glucagon analogs, glucagon derivatives, gastric inhibitory polypeptide (GIP), GIP analogs, GIP derivatives, combined GIP/GLP-1 agonists such as tirzepatide, oxyntomodulin analogs, oxyntomodulin derivatives, therapeutic antibodies, and other suitable therapeutic agents. Drug housing 110 may be constructed of a polymer, metal, ceramic, crystalline solid, or any other material capable of holding the volume of drug 112.

Referring to FIGS. 3 and 5, driving mechanism 108 is positioned at least partially within drug housing 110 and is configured to force the flow of drug 112 from drug housing 110 and through delivery mechanism 104. Driving mechanism 108 illustratively includes a first stopper or piston 136, a second stopper or piston 138, a plunger 140, a cap 142 of housing 110, a driver or biasing member 144, and a trigger 146 (FIG. 3). Driving mechanism 108 is arranged approximately coaxially with longitudinal axis A1. Cap 142 includes an annular shaped end wall having a central opening sized to receive plunger 140. As described herein, plunger 140 and first and second stoppers 136, 138 are movable approximately along axis A1 relative to drug housing 110 towards base 120 as a result of a force exerted by driver 144. Plunger 140 comprises a stopper end 148 positioned adjacent to first stopper 136 and a trigger end 150 positioned adjacent to trigger 146. Plunger 140 is illustratively fixed to first stopper 136 at end 148, although plunger 140 alternatively may be detachably coupled to first stopper 136. Driver 144 is retained between stopper end 148 and an interior surface of cap 142.

Trigger end 150 of plunger 140 is detachably coupled to deployment mechanism 106, illustratively to a hub 152 of deployment mechanism 106, such that plunger 140 moves axially with deployment mechanism 106 relative to drug housing 110 initially during deployment but detaches from hub 152 upon deployment mechanism 106 reaching its end of travel, as further described herein. Deployment mechanism 106 includes a plurality of legs 154, illustratively three legs 154, coupled to hub 152 that extend along drug housing 110 and are approximately parallel to axis A1. Legs 154 are fixedly coupled to a collar 156 of deployment mechanism 106 positioned axially opposite hub 152. Collar 156 may take various forms, such as a closed annular ring, as illustrated, or an open ring. Hub 152, legs 154, and collar 156 of deployment mechanism 106 are illustratively integrally formed but alternatively may comprise one or more discrete components coupled together.

Deployment mechanism 106 and delivery members 122 cooperate to form a deployment interface 158 that facilitates deployment of delivery members 122, illustratively by moving or extending delivery members 122 outwardly relative to drug housing 110 as deployment mechanism 106 moves towards base 120 relative to drug housing 110. In the illustrated embodiment, deployment interface 158 includes a first, annular ramp 160 of collar 156 (FIGS. 4 and 5) that slidably engages second ramps 162 of arms 126. Engagement of ramps 160, 162 during movement of deployment mechanism 106 towards base 120 forces members 122 to extend radially outwardly relative to axis A1 such that penetrating tips 132 engage the intestinal wall. In other embodiments, the collar 156 or the legs 154 may lack a ramp, or deployment interface 158 may take a different form.

In some embodiments, arms 126 may be at least partially constructed of a resilient material to further facilitate deployment. For example, arms 126 may include a shape memory material with time dependent recovery, such as a shape memory polymer, that is compressed during the initial configuration. In this embodiment, the resilient material biases arms 126 outwardly such that upon device 100 entering the deployment configuration and releasing arms 126, as described herein, the arms 126 extend outwardly due to both the radially outward spring force of the resilient arms 126 and the force of deployment mechanism 106 at interface 158. In some embodiments, arms 126 are constructed of a resilient material as described in the previously referenced International Application Publication No. WO2022/060817.

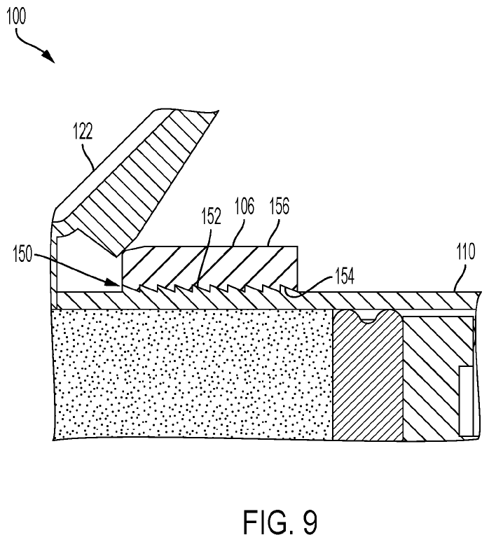

Referring to FIG. 9, device 100 illustratively includes a one-way motion mechanism 150 to permit deployment mechanism 106 to move in a first direction towards base 120 relative to drug housing 110 (to deploy delivery members 122, as described above) and inhibit deployment mechanism 106 from moving in a second, opposite direction away from base 120 relative to drug housing 110 (to inhibit delivery members 122 from moving toward their initial position). In the illustrated embodiment, one-way motion mechanism 150 comprises a ratchet mechanism including one or more ratchet teeth 152 of the drug housing 110 engaged with one or more corresponding ratchet teeth 154 of the collar 156. Corresponding teeth 152, 154 included cooperating angled surfaces that allow motion towards base 120 but block motion of mechanism 106 away from base 120. Other suitable mechanisms may be provided to allow motion in the deployment direction while blocking opposite motion.

With reference to FIG. 5, first stopper 136 and second stopper 138 initially seal drug 112 therebetween and within drug housing 110. As described in further detail herein, once device 100 is deployed first stopper 136 and second stopper 138 subsequently slide approximately along axis A1 due to the force of driver 144. As a result, drug 112 also moves with stoppers 136, 138 and within housing 110. In some embodiments, during deployment stopper 136 pushes drug 112 and in turn drug 112 pushes stopper 138, although stopper 138 may alternatively be fixedly coupled to stopper 136 or plunger 140 to move together. Movement of second stopper 138 past openings in base 120 permits flow of drug 112 from drug housing 110 and into channels 128 of delivery members 122.

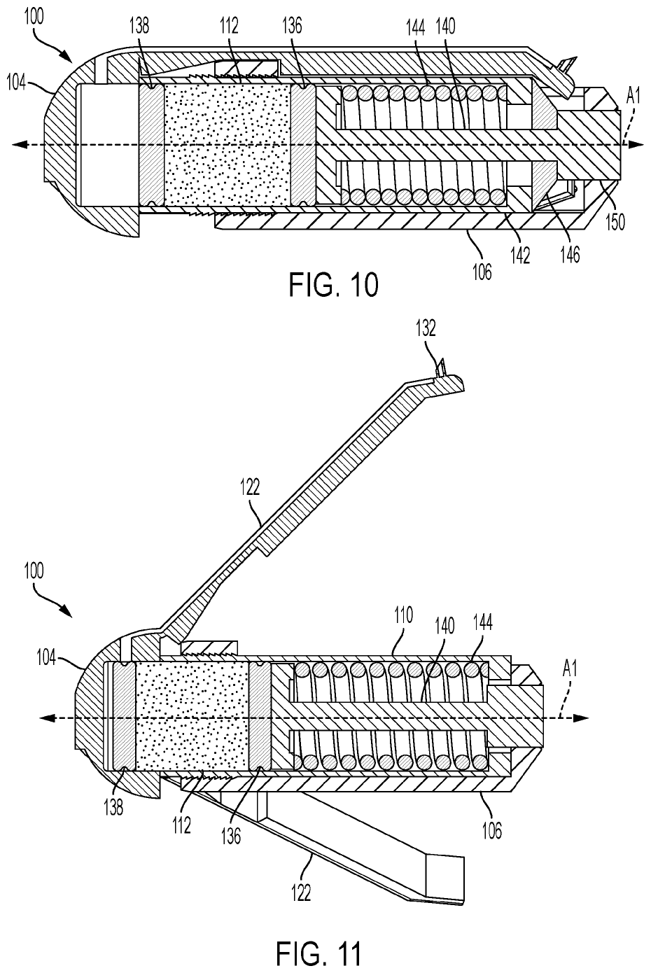

Referring now to FIG. 10, device 100 is illustrated in an intermediate configuration wherein capsule 102 has dissolved upon entering the targeted portion of the GI tract but device 100 is not yet deployed. Driver 144 applies a force (e.g., axial force) that urges plunger 140, first stopper 136, drug 112, and second stopper 138 toward base 120 of delivery mechanism 104. In the initial and intermediate configurations (FIGS. 1, 10), trigger 146 is positioned or wedged between end 150 of plunger 140 and cap 142 of housing 110 to block movement of plunger 140 and prevent end 150 from passing through the opening in cap 142. The presence of trigger 146 therefore also blocks movement of deployment mechanism 106 with plunger 140 still coupled to hub 152. Trigger 146 illustratively has an annular shape with a central opening sized to receive the central portion of plunger 140 but to block end 150. Trigger 146 is constructed of a degradable or dissolvable material, such as a degradable polymer or another suitable degradable material, that degrades or dissolves after a period of time upon being exposed to a GI tract fluid. In an exemplary embodiment, trigger 146 begins to degrade upon capsule 102 separating from device 100 and trigger 146 being exposed to the small intestine fluid.

When trigger 146 degrades sufficiently to be removed from its position between trigger end 150 and cap 142, the device 100 activates and enters a deployment configuration (FIG. 11). In this configuration, the force applied to plunger 140 by driver 144 moves plunger 140 and deployment mechanism 106 together approximately along axis A1 towards base 120. In the illustrated embodiment, deployment mechanism 106 reaches its end of travel upon hub 152 abutting cap 142 of housing 110. Referring to FIG. 11, deployment mechanism 106 engages and moves delivery members 122 outwardly relative to drug housing 110, and device 100 reaches a fully deployed configuration. In some embodiments, delivery members 122 are comprised of a resilient material that further urge members 122 outwardly upon dissolution of trigger 146, as described herein. Penetrating tips 132 of delivery mechanism 104 penetrate the GI tract wall of the patient and anchor drug delivery device 100 to the GI tract wall at the points of penetration. As described herein, deployment mechanism 106 is blocked from backward movement during drug delivery by one-way motion mechanism 150, maintaining interfacing ends 130 against the GI tract wall during delivery. Illustratively, in the configuration of FIG. 11, second stopper 138 continues to block the opening(s) in base 120 to thereby block drug flow into the delivery members 122.

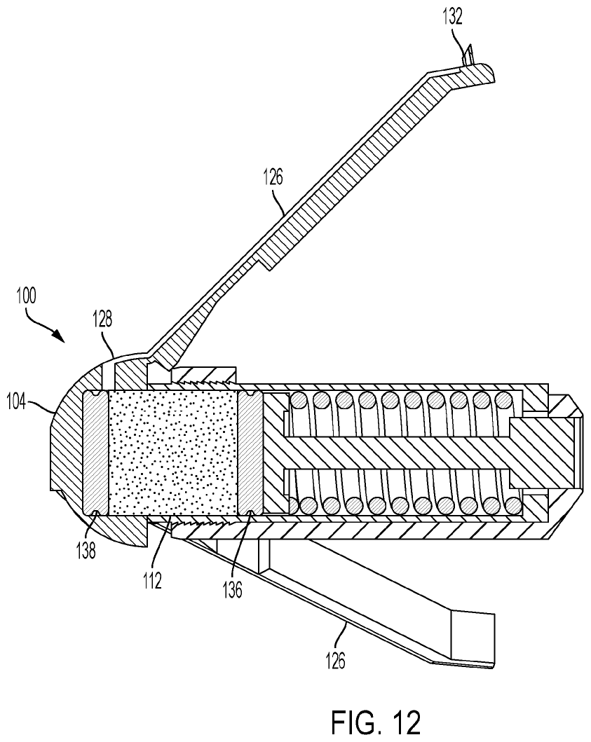

With deployment mechanism 106 at its end of travel, continued application of the force to plunger 140 by driver 144 disengages or decouples plunger 140 from hub 152 and further moves plunger 140 approximately along axis A1, as illustrated in FIG. 12. In one embodiment, end 150 of plunger 140 is friction or interference fit within hub 152, and the force of driver 144 is sufficient to overcome this frictional force upon deployment mechanism 106 reaching its end of travel. Other suitable breakaway features may be provided, such as springs or other mechanisms that facilitate relative movement between plunger 140 and deployment mechanism 106 upon mechanism 106 reaching end of travel.

The continued movement by plunger 140 causes first stopper 136, drug 112, and second stopper 138 to move toward base 120 of delivery mechanism 104. Ultimately and as shown in FIG. 12, second stopper 138 moves past delivery channels 128 of delivery mechanism 104, which allows drug 112 to flow from housing 110 into delivery channels 128 and through arms 126 and penetrating tips 132 for delivery to the patient. With drug 112 delivered to the patient through penetrating tips 132, penetrating tips 132 are configured to degrade after a passage of time. After penetrating tips 132 have degraded past a threshold, device 100 breaks or releases from penetrating tips 132 and passes through the GI tract.

In the illustrated embodiment, driver 144 is a spring that is compressed in the initial and intermediate configurations but is extended during the deployed configuration. In other embodiments, driver 144 may be any suitable mechanism that is capable of delivering a force to move plunger 140 as described herein, including a balloon, compressed gas, a chemical reaction, or a motor. In the illustrated embodiments, first stopper 136, second stopper 138, plunger 140, and driver 144 all remain contained within drug housing 110 due to the presence of cap 142 after drug delivery device 100 has been activated and drug 112 has been delivered. The containment of at least a majority of driving mechanism 108 within drug housing 110 following delivery reduces the likelihood of the components being released into the GI tract. Instead, the components are retained within drug housing 110 which is ultimately passed by the patient along with the rest of device 100.

In some embodiments, instead of including second stopper 138, device 100 alternatively includes one or more different components capable of retaining drug 112 in a sealed manner within drug housing 110 until drug 112 is ready to be introduced to delivery mechanism 104. Such components may include, for example, any of the sealing assemblies and similar components described in the provided in the previously referenced International Application Publication No. WO2022/060817.

In another embodiment, a subset of delivery members 122 (e.g., only one or two members 122 in an embodiment with three total members 122) may contain a delivery channel 128, and accordingly only that subset of delivery members 122 would be configured to deliver drug 112 to the patient. The other delivery member(s) 122 may serve as “dummy” or structural-only delivery members 122 and may be present to assist in securing the drug delivery device 100 within the GI tract without serving as a means for delivering the drug 112. These structural-only delivery members 122 may optionally not include a penetrating tip 132 since no drug 112 would be flowing through the channel, and instead may comprise interfacing features on the interfacing ends 130 to grip the GI tract wall. Such interfacing features may include ridges, protrusions, adhesives, or other gripping/attachment means. Structural delivery members 122 may also comprise microneedles, patches, solid drug deposits, or other drug delivery means to allow diffusion of a drug or other active agent through the wall without penetration.

In some embodiments, drug delivery device 100 may comprise a wireless communication device to send and/or receive signals to/from a wireless receiver, such as a computer, handheld device, a smartphone, or other device. The wireless communication device may measure or sense biological information within the patient after drug delivery device 100 has been ingested. For example, the wireless receiver may send a signal when delivery mechanism 104 has expanded, or when a portion of drug delivery device 100 has degraded. Furthermore, the wireless communication device may measure/sense other biological information within the GI tract, such as chemical concentrations, pH, temperature, or other biological information. The wireless receiver may be used by the patient receiving treatment, or by another user such as a physician or caretaker. The wireless communication device and wireless receiver may communicate through RFID, magneto-acoustics, near field communications, ultrasonic waves, Bluetooth technology, or other suitable wireless communication means.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A drug delivery device comprising:
a capsule configured to degrade within a gastrointestinal tract of a patient;
a drug delivery mechanism disposed within the capsule and configured to interact with a gastrointestinal tract wall of the patient, the drug delivery mechanism comprising a plurality of deployable arms, a plurality of interfacing ends coupled to the plurality of deployable arms, and a plurality of drug delivery channels, wherein the plurality of interfacing ends are in fluid communication with the plurality of drug delivery channels;
a drug housing in fluid communication with the drug delivery mechanism and configured to contain a volume of a drug;
a deployment mechanism coupled to the drug housing, wherein the deployment mechanism is configured to move the plurality of deployable arms from an initial configuration to a deployed configuration following a degradation of the capsule; and
a driving mechanism coupled to the drug housing, the driving mechanism comprising a driver and a stopper, wherein the driver is configured to move the stopper to force the drug through the plurality of drug delivery channels and the plurality of interfacing ends.

2. The drug delivery device of claim 0, wherein the plurality of interfacing ends include a plurality of wall penetrating tips, and a movement of the plurality of deployable arms to the deployed configuration causes the plurality of wall penetrating tips to interact with the gastrointestinal tract wall of the patient.

3. The drug delivery device of claim 1, wherein the driving mechanism is coupled to the deployment mechanism in the initial configuration, and the driving mechanism is further configured to move the deployment mechanism to move the plurality of deployable arms to the deployed configuration.

4. The drug delivery device of claim 1, wherein the driving mechanism further includes a trigger operative to block movement of the driving mechanism and the deployment mechanism in the initial configuration, wherein the trigger is configured degrade upon exposure to a gastrointestinal fluid of the patient.

5. The drug delivery device of claim 4, wherein in response to a degradation of the trigger, the driving mechanism moves the deployment mechanism to extend the plurality of deployable arms to the deployed configuration and forces the delivery of the drug through the plurality of drug delivery channels and the plurality of interfacing ends.

6. The drug delivery device of claim 4, wherein the driving mechanism includes a first end positioned adjacent the trigger and a second end positioned adjacent the stopper, wherein the trigger is positioned between the housing and the first end of the driving mechanism to block movement of the driving mechanism and the deployment mechanism in the initial configuration.

7. The drug delivery device of claim 1, wherein the plurality of interfacing ends include needles configured to pierce the gastrointestinal tract wall of the patient and to degrade within a fluid of the gastrointestinal tract of the patient.

8. The drug delivery device of claim 1, wherein the drug housing extends along a longitudinal axis, and the deployment mechanism moves longitudinally along the drug housing to cause movement of the plurality of deployable arms to the deployed configuration.

9. The drug delivery device of claim 1, wherein the driving mechanism further includes a plunger coupled to the stopper and to the deployment mechanism, wherein the driver drives the plunger and the deployment mechanism to cause movement of the plurality of deployable arms to the deployed configuration, and wherein the driver drives the plunger and the stopper in the drug housing to cause delivery of the drug through the plurality of drug delivery channels and the plurality of interfacing ends.

10. The drug delivery device of claim 9, wherein the driver causes the plunger to decouple from the deployment mechanism following the movement of the plurality of deployable arms to the deployed configuration, and subsequently the driver drives the plunger and the stopper in the drug housing to cause the delivery of the drug through the plurality of drug delivery channels and the plurality of interfacing ends.

11. The drug delivery device of claim 10, wherein the stopper is a first stopper, and the driving mechanism further comprises a second stopper, and the drug is disposed between the first stopper and the second stopper.

12. The drug delivery device of claim 11, wherein the second stopper blocks a flow of the drug through the plurality of drug delivery channels during movement of the plurality of deployable arms to the deployed configuration, and wherein the second stopper is moved by the driving mechanism to allow the flow of the drug through the plurality of drug delivery channels following the movement of the plurality of deployable arms to the deployed configuration.

13. The drug delivery device of claim 1, further comprising a one-way motion mechanism blocking movement of the plurality of deployable arms away from the deployed configuration.

14. The drug delivery device of claim 13, where the one-way motion mechanism comprises a plurality of ratchet teeth.

15. The drug delivery device of claim 1, wherein the plurality of deployable arms are composed of a resilient material configured to urge the plurality of deployable arms towards the deployed configuration.

16. A drug delivery device comprising:
a capsule configured to degrade within a gastrointestinal tract of a patient;
a drug delivery mechanism disposed within the capsule, the drug delivery mechanism comprising a base and a plurality of deployable arms coupled to the base, each deployable arm including a drug delivery channel and an interfacing end in fluid communication with the drug delivery channel, the interfacing end of each deployable arm being configured to interact with a gastrointestinal tract wall of the patient;
a drug housing in fluid communication with the drug delivery mechanism and configured to contain a volume of a drug;
a deployment mechanism including a collar configured to engage the drug delivery mechanism; and
a driving mechanism coupled to the drug housing and to the deployment mechanism, wherein the driving mechanism is operative to move the collar of the deployment mechanism into engagement with the plurality of deployable arms to deploy the plurality of deployable arms, the driving mechanism being further operative to force delivery of the drug through the plurality of drug delivery channels.

17. The drug delivery device of claim 16, wherein the interfacing end of each deployable arm includes a wall penetrating tip, and a deployment of the plurality of deployable arms causes each wall penetrating tip to interact with the gastrointestinal tract wall of the patient.

18. The drug delivery device of claim 16, wherein the driving mechanism includes a trigger configured to degrade within the gastrointestinal tract of the patient, wherein a dissolution of the trigger allows the driving mechanism to move the collar of the deployment mechanism along the drug housing to engage and deploy the plurality of deployable arms, and the driving mechanism forces the delivery of the drug through the plurality of drug delivery channels following the deployment of the plurality of deployable arms.

19. The drug delivery device of claim 18, wherein the driving mechanism decouples from the deployment mechanism following the deployment of the plurality of deployable arms and prior to forcing the delivery of the drug through the plurality of drug delivery channels.

20. The drug delivery device of claim 16, wherein the drug housing extends along a longitudinal axis, the deployment mechanism moves longitudinally along the drug housing to cause deployment of the plurality of deployable arms, and the driving mechanism moves longitudinally along the drug housing to cause delivery of the drug through the plurality of drug delivery channels.
